(19) United States
(12) Patent Application Publication
(10) Pub. No.: US 2022/0125811 A1
(43) Pub. Date: Apr. 28, 2022

(54) INJECTABLE COMPOSITION FOR INDUCING LIPOLYSIS AND METHOD USING THE SAME

(71) Applicant: Eun Hye JUN, Seoul (KR)

(72) Inventor: Eun Hye JUN, Seoul (KR)

(21) Appl. No.: 17/367,430

(22) Filed: Jul. 5, 2021

(30) Foreign Application Priority Data
Oct. 26, 2020 (KR) 10-2020-0139290

(51) Int. Cl. A61K 31/685; A61K 9/00; A61P 3/04; A61K 31/522; A61K 31/385; A61K 31/205; A61K 31/714; A61K 33/00; A61K 38/47; A61K 31/167; A61K 31/4402; A61K 31/137; A61K 31/4172; A61K 31/401; A61K 31/405; A61K 31/198; A61K 31/455; A61K 31/16; A61K 31/525; A61K 31/51; A61K 31/4415

(52) U.S. Cl. CPC A61K 31/685; A61K 31/4415; A61P 3/04; A61K 31/522; A61K 31/385; A61K 31/205; A61K 31/714; A61K 33/00; A61K 38/47; A61K 31/167; A61K 31/4402; A61K 31/137; A61K 31/4172; A61K 31/401; A61K 31/405; A61K 31/198; A61K 31/455; A61K 31/16; A61K 31/525; A61K 31/51; A61K 9/0019

(57) ABSTRACT

Provided is an injectable composition for inducing lipolysis with less pain when injected. Since the composition has an osmolarity within an appropriate range and has a neutral pH, pain at the time of injection of the composition is very small. Also, as a large amount of drug is injected during one treatment, the lipolytic effect is excellent, and local fat removal is possible in a short period of time. The composition may be used safely without side effects. Therefore, the composition may be used in a method for inducing lipolysis.

### CROSS-REFERENCE TO RELATED APPLICATION

This application is based on and claims priority under 35 U.S.C. § 119 to Korean Patent Application No. 10-2020-0139290, filed on Oct. 26, 2020, in the Korean Intellectual Property Office, the disclosure of which is incorporated by reference herein in its entirety.

### BACKGROUND

### 1. Field

One or more embodiments relate to an injectable composition for inducing lipolysis and a method of inducing lipolysis using the composition.

### 2. Description of the Related Art

Localized fat breakdown during weight loss is the biggest concern for those on a diet, especially women who struggle with local fat accumulation. Fat cells in the buttocks and thighs of women have a very high density of adrenergic receptors compared to that of other areas, and since these sites are affected by female hormones, fat cells in these sites become a major concern during weight loss.

Accordingly, interest in lipolysis injection has increased in recent years. Lipolysis injection is a procedure in which several drugs are combined and injected for the purpose of locally decomposing fat cells. For a lipolysis injection the types and mixing ratios of drugs to be combined are very diverse. Lipolysis injection using a needle with a single injection volume of less than 10 cc narrows the injection point intervals and thus small amounts are injected several times. However, when the total amount of the drug is increased to increase the effect, the pain of the patient increases as the patient has to be pierced several times with a needle, and the doctor has to inject the fluid for a long time.

Since lipolysis injection is injected into the fat layer, the pain is generally severe. The pain further increases as the amount of injection solution to be injected increases, and thus there is a limit to increasing the amount of injection solution to maximize the lipolytic effect.

Therefore, there is a need to develop a lipolysis injection capable of alleviating side effects such as pain while increasing an amount of drugs having a lipolytic effect.

### PRIOR ART DOCUMENTS

### Non-Patent Documents

(Non-patent Document 1) Costagliola M et al., “Lipoplasty and Lipolysis: Negative Outcomes and Medicolegal Consequences”, Ann. Plast. Reconstr. Surg. 2017; 1(1): 1003.

### SUMMARY

One or more embodiments include an injectable composition for inducing lipolysis.

One or more embodiments include a method of inducing lipolysis.

Additional aspects will be set forth in part in the description which follows and, in part, will be apparent from the description, or may be learned by practice of the presented embodiments of the disclosure.

According to one or more embodiments, provided is an injectable composition for inducing lipolysis including:

Half saline; an amino acid formulation; choline alfoscerate; aminophylline; lipoic acid; L-carnitine; a multivitamin formulation; hycomin; sodium bicarbonate; hyaluronidase; lidocaine; peniramin; and epinephrine.

The injectable composition for inducing lipolysis may be a composition for lipolysis injection. The injectable composition for inducing lipolysis may be a pharmaceutical composition for lipolysis injection.

In one embodiment, the composition may include:

based on a total weight of the composition, about 1.5 to 2.0 weight % of an amino acid formulation; about 2.0 to 4.0 weight % of choline alfoscerate; about 0.5 to 1.5 weight % of aminophylline; about 1.0 to 2.5 weight % of lipoic acid; about 0.3 to 2.0 weight % of L-carnitine; about 0.26 to 0.43 weight % of a multivitamin formulation; about 0.5 to 1.0 weight % of hycomin; about 0.4 to 1.5 weight % of sodium bicarbonate; about 1300 IU to 1700 IU of hyaluronidase; about 1.0 to 2.5 weight % of lidocaine; about 0.2 to 0.6 weight % of peniramin; and about 0.06 to 0.10 weight % of epinephrine; with the remainder being half saline.

The “half saline” may also be referred to as “physiological saline solution having a sodium chloride concentration of 0.45 weight %”. Unlike common physiological saline has a sodium chloride concentration of 0.9 weight %, half saline is a hypotonic solution.

When free water is used as a solvent in a composition for lipolysis injection, an osmolarity of the composition may be too low, and side effects may occur. When normal saline (0.9% NaCl) is used as a solvent, an osmolarity of the composition may be too high which may cause pain, and a lipolytic effect by a hypotonic solution may not be exhibited. A general lipolysis injection using normal saline is painful.

A volume of the half saline being used may be in a range of about 400 to 600 ml, about 400 to 500 ml, about 450 to 600 ml, about 450 to 500 ml, or about 500 ml. Therefore, in one embodiment, the composition may include 500 ml of half saline and, based on a total weight of the composition, about 1.5 to 2.0 weight % of an amino acid formulation; about 2.0 to 4.0 weight % of choline alfoscerate; about 0.5 to 1.5 weight % of aminophylline; about 1.0 to 2.5 weight % of lipoic acid; about 0.3 to 2.0 weight % of L-carnitine; about 0.26 to 0.43 weight % of a multivitamin formulation; about 0.5 to 1.0 weight % of hycomin; about 0.4 to 1.5 weight % of sodium bicarbonate; about 1300 IU to 1700 IU of hyaluronidase; about 1.0 to 2.5 weight % of lidocaine; about 0.2 to 0.6 weight % of peniramin; and about 0.06 to 0.10 weight % of epinephrine. The composition according to an embodiment includes other ingredients based on 500 ml of half saline, but it is obvious to an ordinary skill in the art to control amounts of physiological saline and other ingredients while maintaining a content ratio or weight of each ingredient.

In one embodiment, a total weight of the composition may be in a range of about 450 to 600 ml, about 450 to about 580 ml, about 500 to 600 ml, about 500 to 580 ml, about 550 to 600 ml, about 550 to 580 ml, about 560 to 600 ml, or about 560 to 580 ml. For example, the composition may be 570.5 ml of an infusion solution. The composition of an amount in these ranges may be injected into a patient by a single treatment. A single treatment refers to a series of procedures injecting the composition into a patient within about 24 hours, for example, injecting the total dose of the composition in a single injection, or injecting the total dose of the composition in several injections (e.g., 2 to 6 divided doses). The composition of an amount within these ranges may not cause inconvenience to the patient's body while maximizing the lipolytic effect.

All the ingredients included in the composition are ingredients that may each be used as a hypodermic injection and thus may not be mineralized when mixed in the physiological saline. The mineralization may refer to a phenomenon in which a precipitate is generated seconds or minutes after being mixed with an infusion solution.

Since all the ingredients included in the composition are ingredients that have a lipolytic effect or may be used as nutrient injections or infusions, the composition may be harmless and safe to the human body.

Concentrations or amounts of all the ingredients in the composition may correspond to ranges safe to the human body.

The composition may be for topical lipolysis injection. Therefore, fat of only a desired site may be decomposed by injecting the composition into a particular site. For example, when the composition is injected into the abdomen, fat in the abdomen is decomposed, but fat in the legs may not be decomposed.

The amino acid formulation may be used to alleviate edema. The edema may be due to interstitial fluid. The edema may be idiopathic edema. Idiopathic edema refers to a condition of chronic edema in the liver, kidney, and other by unidentifiable causes, and most of the idiopathic edema is systemic edema occurring in women in 20s to 40s. Idiopathic edema may generally occur due to lifestyle or posture. Most patients with accumulated fat often complain of edema. Also, as an amount of fat increases, the edema is worsened as well in many cases. In particular, as the amount of fat increases, water having no affinity for fat, that is, water in the interstitial fluid increases, and the swelling may be severely felt. When the amino acid formulation is used, swelling in the body may be reduced.

The amino acid formulation may be used to promote muscle production. Supplementing the body with amino acids may help with protein production. When the muscle mass in the body is insufficient, the basal metabolism decreases compared to a person of the same height and age, and accordingly, the weight may be easily increased, and fat mass may be increased. Therefore, the use of the amino acid formulation may increase muscle mass by promoting muscle production in the body, thereby increasing basal metabolism and reducing fat mass. In addition, the use of the amino acid formulation may help reduce body fat by affecting the adsorption rate of fat metabolism.

The amino acid formulation may include at least one type, for example, 1 type, 2 types, 3 types, 4 types, 5 types, 6 types, 7 types, 8 types, 9 types, 10 types, 11 types, 12 types, 13 types, 14 types, or 15 types, selected from the group consisting of aminoacetic acid, L-alanine, L-arginine, L-histidine, L-isoleucine, L-leucine, L-lysine acetate, L-methionine, L-phenylalanine, L-proline, L-serine, L-threonine, L-valine, N-acetyl-L-cysteine, and L-tryptophan. For example, the amino acid formulation may include aminoacetic acid, L-alanine, L-arginine, L-histidine, L-isoleucine, L-leucine, L-lysine acetate, L-methionine, L-phenylalanine, L-proline, L-serine, L-threonine, L-valine, N-acetyl-L-cysteine, and L-tryptophan.

N-acetyl-L-cystein in the amino acid formulation may exhibit an antioxidant effect.

In one embodiment, the amino acid formulation may include 0.582 g/100 ml of aminoacetic acid, 0.464 g/100 ml of L-alanine, 1.072 g/100 ml of L-arginine, 0.28 g/100 ml of L-histidine, 1.04 g/100 ml of L-isoleucine, 1.309 g/100 ml of L-leucine, 0.971 g/100 ml of L-lysine acetate, 0.11 g/100 ml of L-methionine, 0.088 g/100 ml of L-phenylalanine, 0.573 g/100 ml of L-proline, 0.224 g/100 ml of L-serine, 0.44 g/100 ml of L-threonine, 1.008 g/100 ml of L-valine, 0.07 g/100 ml of N-acetyl-L-cysteine, and 0.07 g/100 ml of L-tryptophan. The amino acid formulation may be Saeronamin Injection (available from Dai Han Pharm), but embodiments are not limited thereto. The amino acid formulation may further include additives such as acetic acid anhydride, sodium edetate hydrate, and water for injection in addition to an amino acid component.

A content of the amino acid formulation may be in a range of about 1.5 to 2.0 weight % or about 1.7 to 2.0 weight % based on the total weight of the composition. When the content of the amino acid formulation is lower than about 1.5 weight %, an osmolarity may be decreased, resulting in prolonged edema or severe pain. The amino acid formulation may be an injection. An amount of the amino acid formulation may be in a range of about 5 to 15 ml, about 5 to 10 ml, or about 10 to 15 ml, but an appropriate amount of the amino acid formulation may be used within these ranges.

The choline alfoscerate may exhibit a lipolytic effect. A content of the choline alfoscerate may be in a range of about 2.0 to 4.0 weight %, about 2.0 to 3.5 weight %, about 2.5 to 4.0 weight %, about 2.5 to 3.5 weight %, or about 2.5 to 3.0 weight % based on the total weight of the composition. The choline alfoscerate may be an injection. An amount of the choline alfoscerate may be in a range of about 11 to 23 ml or about 15 to 20 ml, but an appropriate amount of the choline alfoscerate may be used within these ranges. When the amount of choline alfoscerate increases, the lipolytic effect may be increased, and since the composition according to an embodiment includes choline alfoscerate in a high content, the lipolytic effect of the composition may be excellent.

The aminophylline may be used to assist lipolysis. The aminophylline may promote lipolysis through the following pathways: (1) increasing intracellular cyclic AMP by inhibiting phosphodiesterase type IIIB and inducing protein kinase activation; (2) promoting lipolysis by inhibiting adenosine receptors; and (3) promoting lipolysis by acting as a sympathetic nerve agonist (beta2-adrenergic agonist).

A content of the aminophylline may be in a range of about 0.5 to 1.5 weight %, about 0.5 to 1.2 weight %, or about 0.8 to 1.0 weight based on the total weight of the composition. The aminophylline may be an injection. An amount of the aminophylline may be in a range of about 4 to 10 ml or about 4 to 6 ml, but an appropriate amount of the aminophylline may be used within these ranges. When the amount of the aminophylline is too low, the aminophylline may not be mixed in the composition.

The lipoic acid (or thioctic acid) is also referred to as alpha-lipoic acid, which increases energy consumption by increasing the oxidation of fatty acids and prevents fat accumulation by promoting the absorption of glucose into the skeletal muscle. Also, the lipoic acid may have an antioxidant effect.

A content of the lipoic acid may be in a range of about 1.0 to 2.5 weight %, about 1.0 to 2.0 weight %, about 1.5 to 2.5 weigh %, or about 1.5 to 2.0 weight % based on the total weight of the composition. The lipoic acid may be an injection. An amount of the lipoic acid may be in a range of about 5 to 15 ml or about 8 to 12 ml, but an appropriate amount of the lipoic acid may be used within these ranges.

The L-carnitine may be helpful in the metabolism of fatty acids. The L-carnitine may act to promote collagen production. L-carnitine is a substance that is synthesized from two essential amino acids, lysine and methionine, and thus may promote protein synthesis in muscle and increase glucose utilization in the body.

A content of the L-carnitine may be in a range of about 0.3 to 2.0 weight %, about 0.3 to 1.5 weight %, about 0.3 to 1.0 weight %, about 0.5 to 2.0 weigh %, about 0.5 to 1.5 weight %, about 0.5 to 1.0 weight %, or about 0.8 to 1.0 weight % based on the total weight of the composition. The L-carnitine may be an injection. An amount of the L-carnitine may be in a range of about 2 to 12 ml or about 4 to 6 ml, but an appropriate amount of the L-carnitine may be used within these ranges.

The vitamin formulation may affect an osmolarity of the composition. The vitamin formulation may be a multivitamin formulation. The vitamin formulation may be Beecomhexa Injection (available from Yuhan Corporation). The vitamin formulation may include at least one type, for example, 1 type, 2 types, 3 types, 4 types, 5 types, or 6 types, selected from the group consisting of Nicotinamide, Dexpanthenol, Riboflavin phosphate sodium, Cyanocobalamin, Thiamine hydrochloride, and Pyridoxine hydrochloride. Ingredients of the Beecomhexa Injection may be 40 mg/2 mL of Nicotinamide, 5.17 mg/2 mL of Dexpanthenol, 5.47 mg/2 mL of Riboflavin phosphate sodium, 10 μg/2 mL of Cyanocobalamin, 10 mg/2 mL of Thiamine hydrochloride, and 5 mg/2 mL of Pyridoxine hydrochloride. The vitamin formulation may further include additives such as maleic acid, benzyl alcohol, and sodium hydrogen sulfite in addition to the vitamin ingredients.

A content of the vitamin formulation may be in a range of about 0.26 to 0.43 weight % based on the total weight of the composition. The vitamin formulation may be an injection. An amount of the vitamin formulation may be in a range of about 1.5 to 2.5 ml, but an appropriate amount of the vitamin formulation may be used within this range. When the amount of the vitamin formulation is lower than this range, an osmolarity may be decreased, resulting in prolonged edema or increased pain.

The Hycomin may be used to relieve edema. The Hycomin may be used as an anti-inflammatory analgesic. The Hycomin may be Hycomin Injection (available from Huons). The Hycomin Injection may include 5 mg/2 mL of hydroxocobalamin. The Hycomin Injection may further include additives such as sodium acetate hydrate, acetic acid anhydride, and sodium chloride in addition to hydroxocobalamin.

A content of the Hycomin may be in a range of about 0.5 to 1.0 weight % based on the total weight of the composition. The Hycomin may be an injection. An amount of the Hycomin may be in a range of about 2.5 to 7 ml or about 2.5 to 5 ml, but an appropriate amount of the Hycomin may be used within these ranges.

The sodium bicarbonate may be used to alleviate allergic symptoms. Also, the sodium bicarbonate may be used to control a pH of the composition. When several drugs are mixed with the lipolysis injection solution, a pH of the solution may be lowered, and thus pain during the injection may increase, but the pH may be adjusted to a neutral pH using the sodium bicarbonate to reduce the pain.

A content of the sodium bicarbonate may be in a range of about 0.4 to 1.5 weight %, about 0.4 to 1.2 weight %, about 0.8 to 1.5 weight %, about 0.8 to 1.2 weight %, or about 0.8 to 1.0 weight % based on the total weight of the composition. An amount of the sodium bicarbonate may be in a range of about 2 to 10 ml or about 4 to 6 ml, but an appropriate amount of the sodium bicarbonate may be used within the ranges above. When the amount of the sodium bicarbonate is too low, a pH of the composition may be lowered, and an osmolarity of the composition may be decreased, which may increase pain.

The hyaluronidase promotes lymph circulation, thereby helping the circulation of adipose tissue, reducing adipose tissue, and thus helping to relieve obesity. Lymphedema is caused by the excessive presence of body fluid in the local lymphatic system or by damage to the lymphatic system and thus refers to expansion of tissues resulting from stagnation of interstitial fluid due to the clogged lymphatic system. The hyaluronidase may reduce lymphedema.

An amount of the hyaluronidase may be in a range of about 13000 IU to 1700 IU based on the total weight of the composition. A content of the hyaluronidase may be in a range of about 0.10 to 0.25 weight % or about 0.15 to 0.20 weight % based on the total weight of the composition. However, the hyaluronidase may be excluded when a patient has allergic symptoms such as itching.

The lidocaine may reduce local pain.

A content of the lidocaine may be in a range of about 1.0 to 2.5 weight %, about 1.0 to 2.0 weight %, about 1.5 to 2.5 weight %, or about 1.5 to 2.0 weight % based on the total weight of the composition. The lidocaine may be an injection. An amount of the lidocaine may be in a range of about 6 to 14 ml or about 8 to 12 ml, but an appropriate amount of the lidocaine may be used within these ranges. When the amount of the lidocaine is too low, pain caused by injection of the composition may increase.

The Peniramin may be used to alleviate allergic symptoms. The Peniramin may be Peniramin Injection (available from Yuhan Corporation). The Peniramin Injection may include 4 mg/2 ml of Chlorpheniramine maleate.

A content of the peniramin may be in a range of about 0.2 to 0.6 weight %, about 0.2 to 0.5 weight %, about 0.3 to 0.6 weight %, or about 0.3 to 0.5 weight % based on the total weight of the composition. The Peniramin may be an injection. An amount of the peniramin may be in a range of about 1.5 to 4.0 ml, about 1.5 to 3.0 ml, or about 1.5 to 2.5 ml, but an appropriate amount may be used within these ranges.

The epinephrine (Epinephrine Injection) may reduce local pain.

The epinephrine may serve as a vasoconstrictor. The epinephrine may have an effect of preventing vascular trauma and bruising. In particular, the epinephrine has a vasoconstriction effect, which prevents blood vessel damage and prevents severe bruising caused by an injection.

A content of the epinephrine may be in a range of about 0.06 to 0.10 weight % to about 0.07 to 0.09 weight % based on the total weight of the composition. The epinephrine may be an injection. An amount of the epinephrine may be in a range of about 0.35 to 0.65 ml or about 0.40 to 0.60 ml, but an appropriate amount of the epinephrine may be used within these ranges.

An osmolarity of the compound may be in a range of about 180 to 230 mOsm/L, about 180 to 220 mOsm/L, about 190 to 300 mOsm/L, about 190 to 250 mOsm/L, about 190 to 230 mOsm/L, or about 190 to 220 mOsm/L. For example, an osmolarity of the composition may be about 213 mOsm/L. When the osmolarity of the composition is within these ranges, pain at the injection may be significantly reduced. Generally, most of the patients feel the pain when the lipolysis injection is injected into the fat layer. Causes of pain may be many, for example, pressure at injection of the lipolysis injection, pain due to the injection itself, low pH due to drug mixing, and high osmolarity. Particularly, as an osmolarity of a composition increases, pain at the injection into the body increases, and thus a composition having a high osmolarity may not be used as a lipolysis injection. Also, when the osmolarity of the composition is too high, a lipolysis effect by a hypotonic solution may not be exhibited. When only half saline is used or a component controlling an osmolarity is not added to half saline, the osmolarity of the composition may be very low. An osmolarity of a single half saline is about 154 mOsm/L. However, it has been reported that a half saline or a composition having such a very low osmolarity may cause side effects such as edema, pain, and redness (Costagliola M et al., “Lipoplasty and Lipolysis: Negative Outcomes and Medicolegal Consequences”, Ann. Plast. Reconstr. Surg. 2017; 1(1): 1003). Therefore, the composition for lipolysis injection having an appropriate osmolarity is a very important factor.

The composition may have an appropriate osmolarity within these ranges by adding an amino aid formulation, a vitamin formulation, and sodium bicarbonate in half saline.

The composition may have a neutral pH, for example, pH 7.0 to 8.0. Thus, the composition may cause less pain during injection compared to the lipolysis injection having a low pH.

The composition may further include a pharmaceutically acceptable carrier. For example, when the composition is used as an injection, the composition may be mixed with a buffer, a preservative, a solubilizer, an isotonic agent, and a stabilizer.

A formulation of the composition may be prepared into a variety of dosage forms in combination with the pharmaceutically acceptable carriers described above. For example, when the composition may be prepared as an injection, the formulation may be prepared into single-dose ampule or multidose container.

According to one or more embodiments, provided is a method of inducing lipolysis, the method including administering an effective amount of the injectable composition for inducing lipolysis, according to an embodiment, to a subject in need thereof.

Details of the composition are the same as described above.

The term “effective amount” refers to an amount sufficient to such an extent capable of attaining the aimed effects.

The term “subject” may be an individual in need of the lipolytic effect. The subject may be a mammal, for example, a human, a cow, a horse, a pig, a dog, sheep, a goat, or a cat. In one embodiment, the subject may be a human.

The term “administration” may refer to arrangement of the composition according to an embodiment into a subject through a method or route resulting in at least partial localization of the composition according to an embodiment to the desired site.

The administration may be performed using a method known in the art. The administration may be, for example, direct administration into a subject by any of various routes including intravenous, intramuscular, or subcutaneous administration. The administration may be performed systematically or topically.

The total amount of the composition administered to a subject may be in a range of about 450 to 600 ml, about 450 to 580 ml, about 500 to 600 ml, about 500 to 580 ml, about 550 to 600 ml, about 550 to 580 ml, about 560 to 600 ml, or about 560 to 580 ml. For example, the total amount of the composition administered to a subject may be about 570.5 ml.

Administration frequency may be once or at least twice within the clinically allowable range of side effects. Administration may be given to one site or at least two sites. Administration interval may be daily or from 2 days to 2 weeks, for example, 1 week. If necessary, the same treatment may be repeated after a predetermined period of time. For animals other than humans, a dosage that is the same as that of per kg in a human or a dosage that is determined by, for example, conversion based on the volume ratio (e.g., average value) of organs (e.g., heart) of the target animal and a human, may be administered.

The total amount of the composition may be administered once in a single dose or in 2 to 6 divided doses. When the composition is injected into a large area such as abdomen or thigh, the total amount may be injected in a single dose. When the composition is injected into a narrow area such as forearm or bra line, the total amount may be injected in 2 to 6 divided doses. When the composition is injected in several divided doses, a dose per injection may be about 100 to 150 ml of the composition.

The administration may be an injection using an infusion pump. Since the total amount of the composition exceeds 450 ml, the amount may be difficult to be injected using a syringe. Also, using a syringe may not be appropriate because it requires a very strong pressure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows the results of measuring circumferences of neck, chest, abdomen, and hips after injecting a composition for lipolysis injection prepared in Example 1 each week;

FIGS. 2A to 2D show the results of measuring changes in body weight (2A), changes in body fat (2B), and changes in size of legs (2C) and abdomen (2D) after injecting the composition for lipolysis injection prepared in Example 1 each week;

FIGS. 3A to 3C show the results of measuring changes in body weight (3A), changes in body fat (3B), and changes in size of legs (3C) after injecting the composition for lipolysis injection prepared in Example 1 each week;

FIGS. 4A to 4D show the results of measuring changes in body weight (4A), changes in body fat (4B), changes in size of abdomen (4C), and changes in visceral fat mass (4D) after injecting the composition for lipolysis injection prepared in Example 1 each week;

FIG. 5 shows the results of measuring changes in body weight after injecting the composition for lipolysis injection prepared in Example 1 each week;

FIG. 6 shows the results of measuring changes in body fat after injecting the composition for lipolysis injection prepared in Example 1 each week; and

FIGS. 7A to 7B show images of before (7A) and after (7B) 4 injections of the composition for lipolysis injection prepared in Example 1 over 4 weeks.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present invention will be described in further detail with reference to the following Examples. However, the following Examples are for illustrative purposes and are not intended to limit the scope of the present invention.

### Example 1. Preparation of Composition for Lipolysis Injection

A composition for lipolysis injection was prepared with ingredients and amounts shown in Table 1. In particular, the remaining ingredients were mixed in 0.45% of physiological saline. The amounts were calculated based on the total weight of the composition.

TABLE 1

| Category | Ingredient | Amount | Content |
|---|---|---|---|
| Solvent | 0.45 % of Physiological saline (Half saline; | 500 ml | |
| | including 0.45 % of NaCl) | | |
| Amino acid | 250 ml of Saeronamin Injection (available | 1 g (10 cc) | 1.75 |
| formulation | from Dai Han Pharm) | | weight % |
| Adipocyte | Choline alfoscerate | 16 g (16 cc) | 2.8 |
| decomposer | | | weight % |
| Adipocyte | Aminophylline | 125 mg (5 cc) | 0.88 |
| decomposer | | | weight % |
| Increase in | Lipoic acid or Thioctic acid | 100 mg (10 cc) | 1.75 |
| energy | | | weight % |
| consumption, | | | |
| antioxidant | | | |
| Increase in | L-carnitine | 1 g (5 cc) | 0.88 |
| protein | | | weight % |
| synthesis in | | | |
| muscle, | | | |
| increase in use | | | |
| of glucose | | | |
| Multivitamin | Beecomhexa Injection (available from | 20 mg (2 cc) | 0.35 |
| formulation | Yuhan Corporation) | | weight % |
| Anti- | Hycomin Injection (available from Huons) | 20 mg (4 cc) | 0.7 |
| inflammatory | | | weight % |
| analgesic | | | |
| pH adjusting | Sodium Bicarbonate | 105 mg (5 cc) | 0.88 |
| agent | | | weight % |
| Lymphedema | Hyaluronidase | 1500 IU (1 cc) | 0.18 |
| treatment | | | weight % |
| agent | | | |
| Local | Lidocaine | 40 mg (10 cc) | 1.75 |
| anesthetic | | | weight % |
| Antihistamine | Peniramin Injection (available from Yuhan | 4 mg (2 cc) | 0.35 |
| | Corporation) | | weight % |
| Vasoconstrictor | Epinephrine Injection | 0.5 mg (0.5 cc) | 0.08 |
| | | | weight % |

In Table 1, ingredients of Saeronamin Injection are 0.582 g/100 ml of aminoacetic acid, 0.464 g/100 ml of L-alanine, 1.072 g/100 ml of L-arginine, 0.28 g/100 ml L-histidine, 1.04 g/100 ml of L-isoleucine, 1.309 g/100 ml of L-leucine, 0.971 g/100 ml of L-lysine acetate, 0.11 g/100 ml of L-methionine, 0.088 g/100 ml of L-phenylalanine, 0.573 g/100 ml of L-proline, 0.224 g/100 ml of L-serine, 0.44 g/100 ml of L-threonine, 1.008 g/100 ml of L-valine, 0.07 g/100 ml of N-acetyl-L-cystein, and 0.07 g/100 ml of L-tryptophan.

Ingredients of Beecomhexa Injection are 40 mg/2 mL of nicotinic acid amide, 5.17 mg/2 mL of dexpanthenol, 5.47 mg/2 mL of riboflavin phosphate sodium, 10 μg/2 mL of cyanocobalamin, 10 mg/2 mL of thiamine hydrochloride, and 5 mg/2 mL of pyridoxine hydrochloride.

An ingredient of Hycomin Injection is 5 mg/2 ml of hydroxocobalamin.

An ingredient of Peniramin Injection is 4 mg/2 ml of chlorpheniramine maleate.

### Experimental Example 1: Calculation of Osmolarity

An osmolarity of the composition for lipolysis injection prepared in Example 1 was calculated. Calculation of osmolarity usually matters whether the material is ionizable in an infusion solution. In Table 1, the half saline, vitamin, sodium bicarbonate, and amino acid correspond to ingredients capable of changing the osmolarity of the composition. The rest of the ingredients may slightly raise or lower the osmolarity, but the effect is mostly considered insignificant, and medically these drugs are not included in calculation of an osmolarity of an infusion solution.

1) Half saline is a solution including 77 mOsm of NaCl in pure water.

2) A 5 mEq (milliequivalent) osmolarity of sodium bicarbonate (8.4%) is 10 mOsm.

3) An osmolarity of 6 cc of multivitamin formulation is 24.7 mOsm.

4) An osmolarity of 10 cc of amino acid formulation is 10 mOsm.

5) The total amount of the composition of Example 1 is 570.5 mL.

Therefore, an osmolarity (mOsm/L) of the composition of Example 1 is (77+10+24.7+10) mOsm/570.5 mL=213 mOsm/L.

Experimental Example 2: Confirmation of lipolytic effect

(1) Case 1

The composition for lipolysis injection of Example 1 was injected into the thigh of a woman in early 40s once a week for a total of 6 injections in 6 weeks. For each injection, the total amount of the composition for lipolysis injection of Example 1 was all injected using an infusion pump. After 6 weeks, the body weight of the woman decreased from 54.7 kg (25.5% of body fat percentage) to 51.7 kg (20.2% of body fat percentage), and the circumference size of the upper thigh decreased from 54 cm to 49 cm. Thereafter, the shape of the convex area of the thigh did not return even when the weight increased again.

(2) Case 2

The composition for lipolysis injection of Example 1 was injected into the buttocks of a woman in early 30s once a week for a total of 5 injections in 5 weeks. For each injection, the total amount of the composition for lipolysis injection of Example 1 was all injected using an infusion pump. Body composition was analyzed using InBody. InBody is an excellent device that shows where the measurer's statuses such as muscle mass, fat mass, and the size of a particular part of the body are at compared to the average based on the body data such as heights and weights of Koreans.

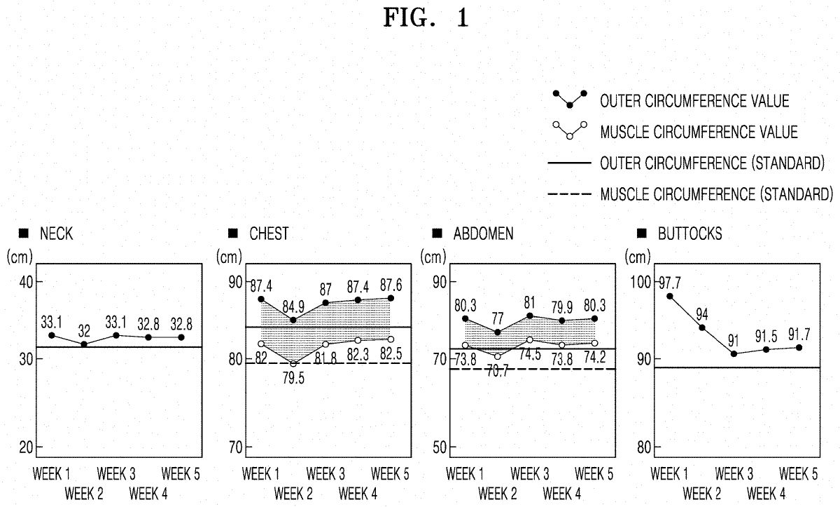

FIG. 1 shows the results of measuring circumferences of neck, chest, abdomen, and hip after injecting the composition for lipolysis injection of Example 1 each week.

As shown in FIG. 1, the patient did not lose weight for 5 weeks due to the lack of diet control, but the size of the hip circumference after the injection of the composition for lipolysis injection was reduced by 6 cm. No particular side effects were found, and satisfaction of the patient was high as the injections were effective in relieving fatigue.

(3) Case 3

The patient was a 33-year-old female with a central obesity type with long and thin legs and arms and a lot of fat in the abdomen. The patient wanted to reduce the size of the abdomen without changing the size of the legs. The composition for lipolysis injection of Example 1 was injected into the abdomen of the patient once a week for a total of 5 injections in 5 weeks. For each injection, the total amount of the composition for lipolysis injection of Example 1 was all injected using an infusion pump.

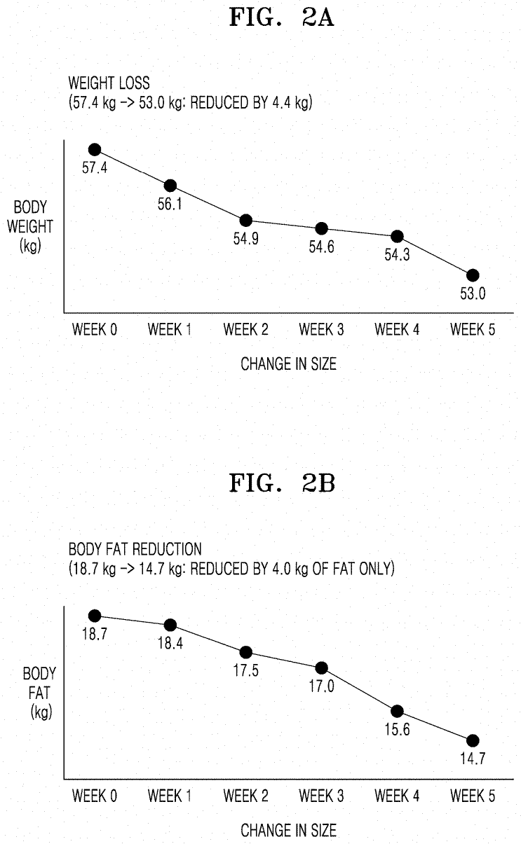

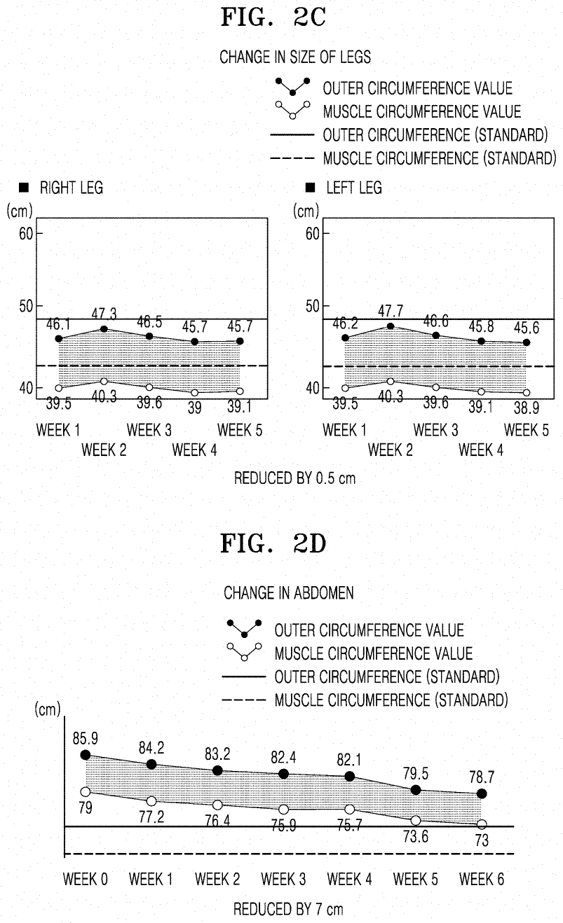

FIGS. 2A to 2D show the results of measuring changes in body weight (2A), changes in body fat (2B), and changes in size of legs (2C) and abdomen (2D) after injecting the composition for lipolysis injection prepared in Example 1 each week.

As shown in FIGS. 2A to 2D, the graphs related to the size of legs and abdomen have a baseline, which refers to the average size of legs and abdomen of Koreans having the same height and weight. That is, the patient had a body type of a Westerner having a thicker abdominal circumference and slender leg circumferences as compared with other Koreans of the same height and weight. However, the sizes of legs and other sites where the fat was not desired to be removed without injection of the composition for lipolysis injection for 5 weeks were almost unchanged, and only the size of abdomen, the desired site, significantly decreased. The weight loss for 5 weeks was 4.4 kg, of which fat mass decreased by 4 kg, and there was almost no muscle loss.

Therefore, it was confirmed that the composition for lipolysis injection of Example 1 has an excellent effect as a local lipolysis injection and overall lipolysis effects.

(4) Case 4

The patient was a 20-year-old female who did not lose her leg size while losing more than 13 kg of weight through diet management and thus wanted to reduce the size of her legs. The composition of lipolysis injection of Example 1 was injected into the thighs of the patient once a week for a total of 6 injections in 6 weeks. For each injection, the total amount of the composition for lipolysis injection of Example 1 was all injected using an infusion pump.

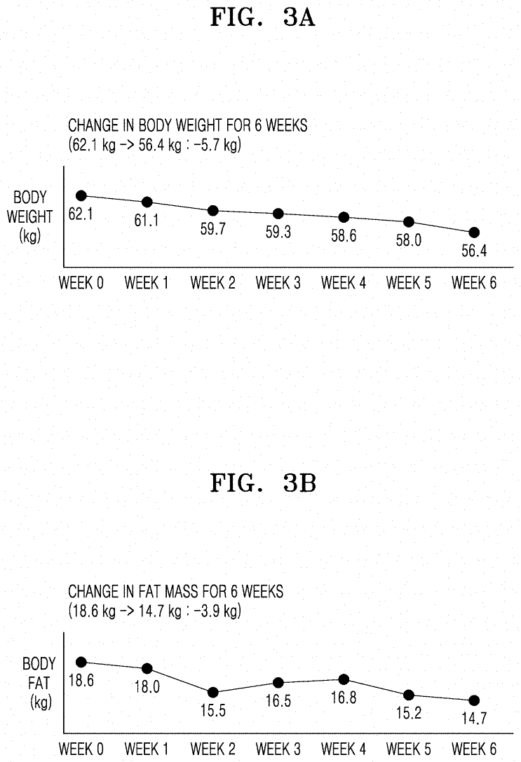

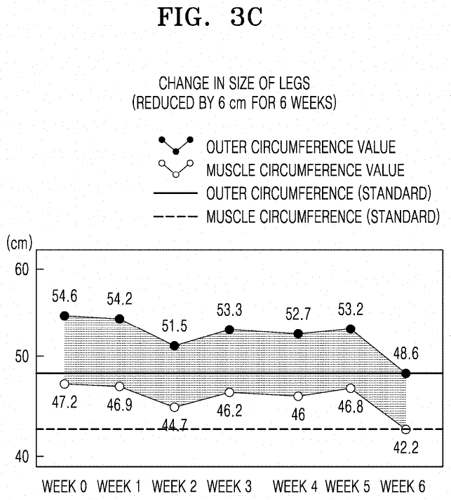

FIGS. 3A to 3C show the results of measuring changes in body weight (3A), changes in body fat (3B), and changes in size of legs (3C) after injecting the composition for lipolysis injection prepared in Example 1 each week.

As shown in FIGS. 3A to 3C, the size of thighs was reduced by 6 cm and the abdominal size by 1.5 cm for 6 weeks as a result of injecting the composition for lipolysis injection.

(5) Case 5

The patient was a 42-year-old male who wanted to lose abdominal visceral fat and abdominal fat after being diagnosed with hypertension and diabetes. The composition for lipolysis injection of Example 1 was injected into the abdomen of the patient once a week for a total of 6 injections in 6 weeks. For each injection, the total amount of the composition for lipolysis injection of Example 1 was all injected using an infusion pump.

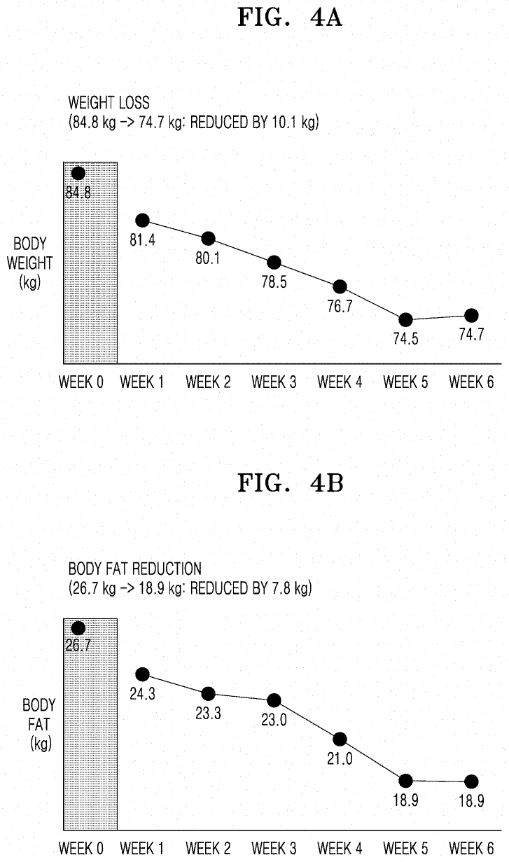

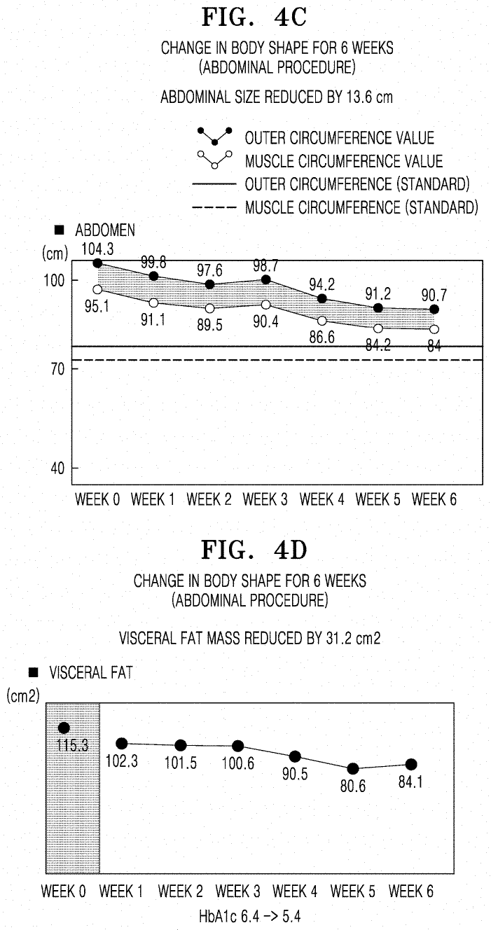

FIGS. 4A to 4D show the results of measuring changes in body weight (4A), changes in body fat (4B), changes in size of abdomen (4C), and changes in visceral fat mass (4D) after injecting the composition for lipolysis injection prepared in Example 1 each week.

As shown in FIGS. 4A to 4D, the abdominal size was reduced by 13.6 cm in 6 weeks, and the body weight and body fat mass were also decreased significantly. Meanwhile, diabetes, hyperlipidemia, and hypertension of the patient have been normalized. The body weight was almost maintained, and the abdominal size was also maintained within 1 cm almost without change even after 6 months.

(6) Case 6

The patient was a 49-year-old female who had been taking obesity drugs for 3 years, and she had the diabetes, hyperlipidemia, and hypertension which had been worsened for 5 years. Thus, the patient wanted health care and abdominal fat reduction. The composition for lipolysis injection of Example 1 was injected into the abdomen of the patient once a week for a total of 17 injections in 17 weeks. For each injection, the total amount of the composition for lipolysis injection of Example 1 was all injected using an infusion pump.

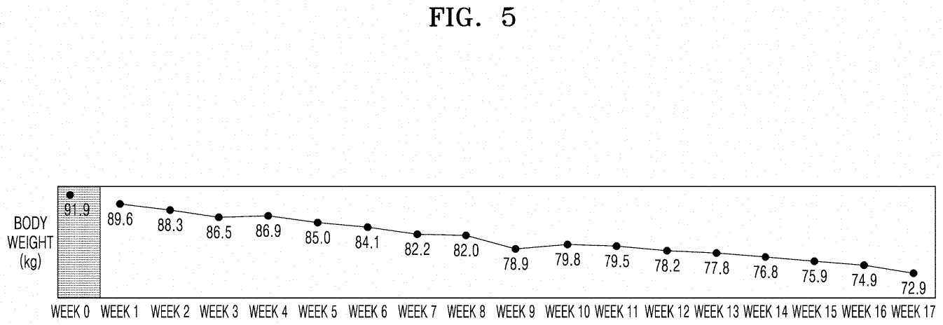

FIG. 5 shows the results of measuring changes in body weight after injecting the composition for lipolysis injection of Example 1 each week.

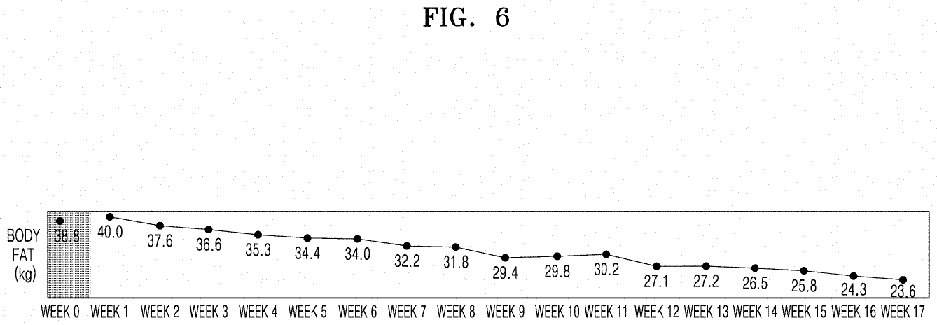

FIG. 6 shows the results of measuring changes in body fat after injecting the composition for lipolysis injection of Example 1 each week.

As shown in FIGS. 5 and 6, the body weight and body fat steadily decreased without a single stagnation period in 17 weeks. Also, 3 months after the start of the procedure, the levels of diabetes and hyperlipidemia were almost normalized, and thus the drug for hyperlipidemia was discontinued. After 2 months from then on, hypertension, hyperlipidemia, and diabetes of the patient were completely within the normal range, and thus the patient stopped all the drugs and started exercise and maintenance therapy.

(7) Case 7

The patient was a woman in late 50s who wanted to lose abdominal fat. The composition for lipolysis injection of Example 1 was injected into the abdomen of the patient once a week for a total of 4 injections in 4 weeks. For each injection, the total amount of the composition for lipolysis injection of Example 1 was all injected using an infusion pump.

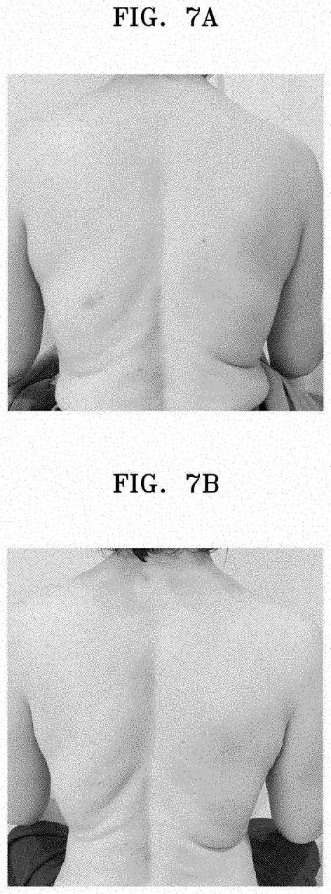

FIGS. 7A to 7B show images of before (7A) and after (7B) 4 injections of the composition for lipolysis injection prepared in Example 1 over 4 weeks.

As shown in FIGS. 7A to 7B, the waist line became slim due to the reduced abdominal fat, and the weight was also reduced by about 5 kg.

In addition to the above cases, dozens of patients of various ages and sexes, from 18-year-old female students to 75-year-old men, were treated with the composition for lipolysis injection of Example 1, and there were no side effects.

Therefore, it was confirmed that the composition for lipolysis injection according to an embodiment safely exhibits a local lipolysis effect of only a desired area without side effects.

According to an embodiment, an injectable composition for inducing lipolysis has an osmolarity within an appropriate range and a neutral pH, and thus injection of the composition is less painful. Also, since an amount of the drug being injected in a single procedure is a lot, the lipolytic effect of the composition is excellent, which makes local fat loss possible in a short period of time. The composition may be used safely without side effects. Therefore, the composition may be used in a method of inducing lipolysis.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An injectable composition for inducing lipolysis, the composition comprising:
based on a total weight of the composition, about 1.5 to 2.0 weight % of an amino acid formulation; about 2.0 to 4.0 weight % of choline alfoscerate; about 0.5 to 1.5 weight % of aminophylline; about 1.0 to 2.5 weight % of lipoic acid; about 0.3 to 2.0 weight % of L-carnitine; about 0.26 to 0.43 weight % of a multivitamin formulation; about 0.5 to 1.0 weight % of hycomin; about 0.4 to 1.5 weight % of sodium bicarbonate; about 1300 IU to 1700 IU of hyaluronidase; about 1.0 to 2.5 weight % of lidocaine; about 0.2 to 0.6 weight % of peniramin; and about 0.06 to 0.10 weight % of epinephrine; with the remainder being half saline,
wherein the amino acid formulation comprises aminoacetic acid, L-alanine, L-arginine, L-histidine, L-isoleucine, L-leucine, L-lysine acetate, L-methionine, L-phenylalanine, L-proline, L-serine, L-threonine, L-valine, N-acetyl-L-cysteine, and L-tryptophan,
the multivitamin formulation comprises nicotinic acid amide, dexpanthenol, riboflavin sodium phosphate, cyanocobalamin, thiamine hydrochloride, and pyridoxine hydrochloride, and
an osmolarity of the composition is in a range of about 180 to 230 mOsm/L.

2. The composition of claim 1, wherein an amount of the half saline in the composition is in a range of about 400 to 500 ml.

3. The composition of claim 1, wherein a pH of the composition is in a range of about 7.0 to 8.0.

4. A method of inducing lipolysis, the method comprising administering an effective amount of the composition of claim 1 to a subject in need thereof.

5. The method of claim 4, wherein the total amount of the composition administered is in a range of about 450 to 600 ml.

6. The method of claim 5, wherein the total amount of the composition is administered once in a single dose or in 2 to 6 divided doses.

7. The method of claim 4, wherein the administering of the composition is an injection using an infusion pump.
